(19) United States
(12) Patent Application Publication
(10) Pub. No.: US 2005/0186579 A1
(43) Pub. Date: Aug. 25, 2005

(54) MALDI-MS analysis of nucleic acids bound to a surface

(76) Inventors: Douglas J. Dellinger, Boulder, CO (US); Barry E. Boyes, Wilmington, DE (US); Gordon R. Nicol, Middletown, DE (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/785,337

(22) Filed: Feb. 23, 2004

(51) Int. Cl. C12Q 1/68

(52) U.S. Cl. 435/6

(57) ABSTRACT

A method of analyzing a polynucleotide using matrix assisted laser desorption/ionization mass spectrometry (MALDI-MS) is described. The method includes obtaining the polynucleotide bound to a substrate via a linker moiety having a triaryl methyl linker group. The polynucleotide bound to the substrate is then contacted with a matrix material and analyzed by MALDI-MS. During the MALDI-MS analysis, laser radiation is directed at the matrix material, thereby exciting the matrix material and causing cleavage of the linker moiety. Ions generated as a result of this excitation and cleavage process are then analyzed to provide information about the polynucleotide.

Related Applications: Related subject matter is disclosed in a U.S. patent application entitled “Quality Control Method for Array Manufacture” filed concurrently with the present application by Dellinger et al., and also in U.S. Patent Applications entitled “Method of Polynucleotide Synthesis Using Modified Support”, Ser. No. 10/652,049, filed by Dellinger et al. on Aug. 30, 2003; and “Cleavable Linker for Polynucleotide Synthesis”, Ser. No. 10/652,063, filed by Dellinger et al. on Aug. 30, 2003; all of which are incorporated herein by reference in their entireties, provided that, if a conflict in definition of terms arises, the definitions provided in the present application shall be controlling.

This invention was made with Government support under Agreement No. N39998-01-9-7068. The Government has certain rights in the invention.

### DESCRIPTION

1. Field of the Invention

The invention relates generally to analysis of nucleic acids. More particularly, the invention relates to MALDI-MS analysis of nucleic acids immobilized on a surface via a cleavable linker moiety.

2. Background of the Invention

Over the past twenty years, the method of choice for the chemical synthesis of oligodeoxynucleotides (ODNs) has been the phosphoramidite four-step process which utilizes the reaction of deoxynucleoside phosphoramidites with a solid phase tethered deoxynucleoside or oligodeoxynucleotide. This process is illustrated schematically in FIG. 1 (wherein “B” typically represents a purine or pyrimidine base, “DMT” represents dimethoxytrityl, “iPR” represents isopropyl, and “●-” represents the growing polynucleotide strand bound to the solid phase). See Letsinger, R. L. et al.; J. Am. Chem. Soc. (1976) 98: 3655-61; Beaucage, S. L. et al.; Tetrahedron Lett. (1981) 22: 1859-62; and Matteucci, M. D., et al.; J. Am. Chem. Soc. (1981) 103: 3186-91. In the first step (“deprotection”) of the four-step cycle, the 5′-O-dimethoxytrityl (DMT) group is removed from a deoxynucleoside linked to the polymer support. Step 2 (“condensation”), elongation of a growing oligodeoxynucleotide, occurs via the initial formation of a phosphite triester internucleotide bond. This reaction product is first treated with a capping agent (step 3—“capping”) designed to esterify failure sequences and cleave phosphite reaction products on the heterocyclic bases. The nascent phosphite internucleotide linkage is then oxidized to the corresponding phosphotriester (step 4—“oxidation”). The synthesis then continues with the deprotection step, in which the DMT group is removed from the growing oligodeoxynucleotide using a large excess of a weak acid, such as trichloroacetic acid (TCA), in an organic solvent. Further repetitions of this four-step process generate the ODN of desired length and sequence. The final product is cleaved from the solid phase and obtained free of base and the b-cyanoethylphosphate protecting groups (see Ogilvie, K. K., et al.; Can. J. Chem. (1980) 58: 2686-93; Sinha, N. D., et al.; Tetrahedron Lett. (1983) 24: 5843-46) by treatment of the support with concentrated ammonium hydroxide. See Matteucci, M. D., et al.; J. Am. Chem. Soc. (1981) 103: 3186-91. ODNs synthesized with this chemistry continue to be of satisfactory quality for most biological uses such as DNA sequencing, PCR applications, and site-specific mutagenesis.

An improved method of polynucleotide synthesis has been reported whereby the oxidation and deprotection reactions are performed simultaneously using a mildly basic solution of peroxy anions (FIG. 2). See U.S. Pat. No. 6,222,030 and U.S. patent application Ser. No. 09/916,369 filed Jul. 27, 2001. See also Sierzchala, A. B., et al., J. Am. Chem. Soc. (2003) in press. For this new synthesis approach, the trityl protecting group typically used for the monomers in the traditional four-step phosphoramidite-based synthesis (FIG. 1) is not used, and further reports of the new synthesis approach have described the use of trityl group chemistry for providing a cleavable linker for surface attachment. See U.S. patent application Ser. No. 10/652,063 filed Aug. 30, 2003 by Dellinger et al.

Whether chemically synthesized or isolated from biological sources, polynucleotides typically need to be analyzed to obtain specific information, such as size, purity, identity, etc. Typical means of analysis include chromatographic or electrophoretic separations, chemical analyses, specific enzymatic cleavage reactions, and other means. An additional method of analyzing polynucleotides that can be used to provide specific information about the polynucleotides would be helpful to researchers in the field.

### SUMMARY OF THE INVENTION

We have now developed a method of analyzing a polynucleotide using matrix assisted laser desorption/ionization mass spectrometry (MALDI-MS). The method includes obtaining the polynucleotide bound to a substrate via a linker moiety having a triaryl methyl linker group. The polynucleotide bound to the substrate is then contacted with a matrix material and analyzed by MALDI-MS. During the MALDI-MS analysis, laser radiation is directed at the matrix material, thereby exciting the matrix material and causing cleavage of the linker moiety. Ions generated as a result of this excitation and cleavage process are then analyzed to provide information about the polynucleotide.

In the method of the present invention, polynucleotide bound to the substrate may be obtained by binding the polynucleotide onto the substrate. The polynucleotide may be bonded to the substrate via a triaryl methyl linker group that covalently links the substrate to the polynucleotide. Alternatively, the polynucleotide may be provided already bound to the substrate via a triaryl methyl linker group.

The invention provides a method of analyzing a polynucleotide using matrix assisted laser desorption/ionization mass spectrometry (MALDI-MS). In an embodiment, the method includes obtaining a composition having the structure (I)
●-Cgp-Trl-Cgp′-Pnt (I)
wherein the groups are defined as follows:
●- is a substrate,
Trl is a triaryl methyl linker group having three aryl groups, each bound to a central methyl carbon, at least one of said three aryl groups having one or more substituents,
Cgp is a linking group linking the substrate and the triaryl methyl linker group, or is a bond linking the substrate and the triaryl methyl linker group,
Pnt is a polynucleotide, and
Cgp′ is a linking group linking the polynucleotide and the triaryl methyl linker group, or is a bond linking the polynucleotide and the triaryl methyl linker group.
   The composition having the structure (I) is then contacted with a matrix material and analyzed by MALDI-MS. During the MALDI-MS analysis, laser radiation is directed at the matrix material, thereby exciting the matrix material and releasing the polynucleotide from the substrate. Ions generated as a result of this excitation and release process are then analyzed to provide information about the polynucleotide.

The methods of analyzing a polynucleotide, triaryl methyl linker groups, and compositions having the structure (I) are further described herein. Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the materials and methods particularly pointed out in the appended claims.

### BRIEF DESCRIPTION OF THE DRAWINGS

These and other features of the invention will be understood from the description of representative embodiments of the method herein and the disclosure of illustrative materials for carrying out the method, taken together with the Figures, wherein

FIG. 1 schematically illustrates prior art synthesis of polynucleotides.

FIG. 2 depicts a prior art synthesis scheme for synthesizing polynucleotides, the synthesis scheme employing a two step synthesis cycle, including a coupling step and a simultaneous deprotection and oxidation step.

FIG. 3 shows an embodiment in accordance with the present invention, in which a polynucleotide is released from a substrate in a MALDI-MS analysis method.

FIG. 4 shows an embodiment in accordance with the present invention, in which a phosphoramidite is coupled to a substrate.

FIG. 5 give shows mass spectra resulting from a MALDI-MS analysis, described herein.

FIG. 6 shows mass spectra prepared in accordance with the present invention.

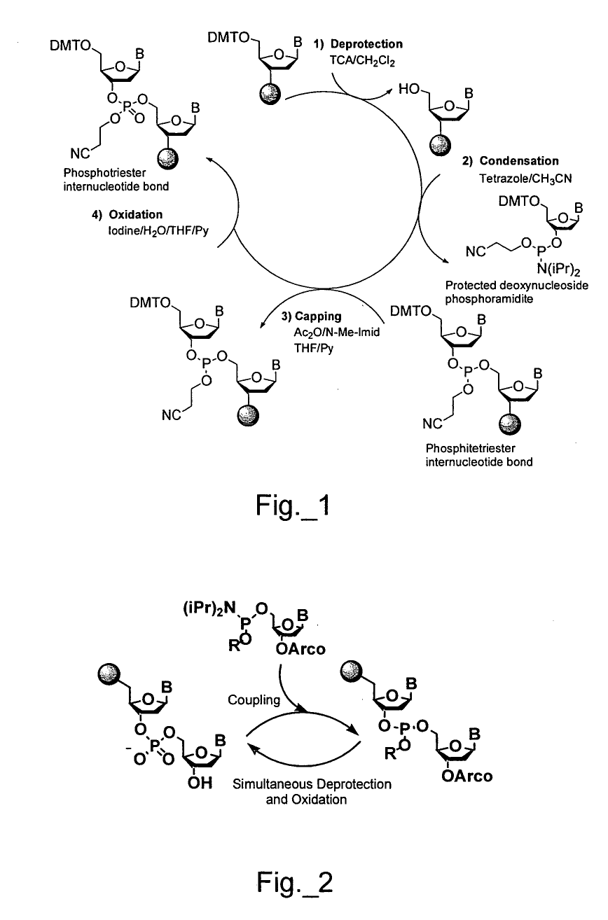

To facilitate understanding, identical reference numerals/designations have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “a solid support” includes a plurality of insoluble supports. Likewise, reference to “a polynucleotide” includes embodiments having a plurality of polynucleotides. Similarly, reference to “a substituent”, as in a compound substituted with “a substituent”, includes the possibility of substitution with more than one substituent, wherein the substituents may be the same or different. In this specification and in the claims that follow, reference will be made to a number of terms that shall be defined to have the following meanings unless a contrary intention is apparent.

A “nucleotide” refers to a sub-unit of a nucleic acid (whether DNA or RNA or analogue thereof) which includes a phosphate group, a sugar group and a heterocyclic base, as well as analogs of such sub-units. A “nucleoside” references a nucleic acid subunit including a sugar group and a heterocyclic base. A “nucleoside moiety” refers to a portion of a molecule having a sugar group and a heterocyclic base (as in a nucleoside); the molecule of which the nucleoside moiety is a portion may be, e.g. a polynucleotide, oligonucleotide, or nucleoside phosphoramidite. A “nucleotide monomer” refers to a molecule which is not incorporated in a larger oligo- or poly-nucleotide chain and which corresponds to a single nucleotide sub-unit; nucleotide monomers may also have activating or protecting groups, if such groups are necessary for the intended use of the nucleotide monomer. A “polynucleotide intermediate” references a molecule occurring between steps in chemical synthesis of a polynucleotide, where the polynucleotide intermediate is subjected to further reactions to get the intended final product, e.g. a phosphite intermediate which is oxidized to a phosphate in a later step in the synthesis, or a protected polynucleotide which is then deprotected. An “oligonucleotide” generally refers to a nucleotide multimer of about 2 to 200 nucleotides in length, while a “polynucleotide” includes a nucleotide multimer having at least two nucleotides and up to several thousand (e.g. 5000, or 10,000) nucleotides in length. It will be appreciated that, as used herein, the terms “nucleoside”, “nucleoside moiety” and “nucleotide” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., methylated purines or pyrimidines, acylated purines or pyrimidines, and the like, or the addition of a protecting group such as acetyl, difluoroacetyl, trifluoroacetyl, isobutyryl, benzoyl, or the like. The purine or pyrimidine base may also be an analog of the foregoing; suitable analogs will be known to those skilled in the art and are described in the pertinent texts and literature. Common analogs include, but are not limited to, 1-methyladenine, 2-methyladenine, N6-methyladenine, N6-isopentyladenine, 2-methylthio-N-6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

The term “alkyl” as used herein, unless otherwise specified, refers to a saturated straight chain, branched or cyclic hydrocarbon group of 1 to 24, typically 1-12, carbon atoms, such as methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “lower alkyl” intends an alkyl group of one to six carbon atoms, and includes, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “cycloalkyl” refers to cyclic alkyl groups such as cyclopropyl, cyclobutyl, cyclopentyl, cyclohexyl, cycloheptyl and cyclooctyl.

The term “modified alkyl” refers to an alkyl group having from one to twenty-four carbon atoms, and further having additional groups, such as one or more linkages selected from ether-, thio-, amino-, phospho-, oxo-, ester-, and amido-, and/or being substituted with one or more additional groups including lower alkyl, aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. The term “modified lower alkyl” refers to a group having from one to six carbon atoms and further having additional groups, such as one or more linkages selected from ether-, thio-, amino-, phospho-, keto-, ester- and amido-, and/or being substituted with one or more groups including lower alkyl; aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. The term “alkoxy” as used herein refers to a substituent —O—R wherein R is alkyl as defined above. The term “lower alkoxy” refers to such a group wherein R is lower alkyl. The term “thioalkyl” as used herein refers to a substituent —S—R wherein R is alkyl as defined above.

The term “alkenyl” as used herein, unless otherwise specified, refers to a branched, unbranched or cyclic (e.g. in the case of C₅ and C₆) hydrocarbon group of 2 to 24, typically 2 to 12, carbon atoms containing at least one double bond, such as ethenyl, vinyl, allyl, octenyl, decenyl, and the like. The term “lower alkenyl” intends an alkenyl group of two to six carbon atoms, and specifically includes vinyl and allyl. The term “cycloalkenyl” refers to cyclic alkenyl groups.

The term “alkynyl” as used herein, unless otherwise specified, refers to a branched or unbranched hydrocarbon group of 2 to 24, typically 2 to 12, carbon atoms containing at least one triple bond, such as acetylenyl, ethynyl, n-propynyl, isopropynyl, n-butynyl, isobutynyl, t-butynyl, octynyl, decynyl and the like. The term “lower alkynyl” intends an alkynyl group of two to six carbon atoms, and includes, for example, acetylenyl and propynyl, and the term “cycloalkynyl” refers to cyclic alkynyl groups.

The term “aryl” as used herein refers to an aromatic species containing 1 to 5 aromatic rings, either fused or linked, and either unsubstituted or substituted with one or more substituents typically selected from the group consisting of lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxylthio, mercapto, amino, imino, halo, cyano, nitro, nitroso, azido, carboxy, sulfide, sulfone, sulfoxy, phosphoryl, silyl, silyloxy, and boronyl. Typical aryl groups contain 1 to 3 fused aromatic rings, and more typical aryl groups contain 1 aromatic ring or 2 fused aromatic rings. Aromatic groups herein may or may not be heterocyclic. The term “aralkyl” intends a moiety containing both alkyl and aryl species, typically containing less than about 24 carbon atoms, and more typically less than about 12 carbon atoms in the alkyl segment of the moiety, and typically containing 1 to 5 aromatic rings. The term “aralkyl” will usually be used to refer to aryl-substituted alkyl groups. The term “aralkylene” will be used in a similar manner to refer to moieties containing both alkylene and aryl species, typically containing less than about 24 carbon atoms in the alkylene portion and 1 to 5 aromatic rings in the aryl portion, and typically aryl-substituted alkylene. Exemplary aralkyl groups have the structure —(CH₂)ⱼ—Ar wherein j is an integer in the range of 1 to 24, more typically 1 to 6, and Ar is a monocyclic aryl moiety.

The term “heterocyclic” refers to a five- or six-membered monocyclic structure or to an eight- to eleven-membered bicyclic structure which is either saturated or unsaturated. The heterocyclic groups herein may be aliphatic or aromatic. Each heterocyclic group consists of carbon atoms and from one to four heteroatoms selected from the group consisting of nitrogen, oxygen and sulfur. As used herein, the term “nitrogen heteroatoms” includes any oxidized form of nitrogen and the quaternized form of nitrogen. The term “sulfur heteroatoms” includes any oxidized form of sulfur. Examples of heterocyclic groups include purine, pyrimidine, piperidinyl, morpholinyl and pyrrolidinyl. “Heterocyclic base” refers to any natural or non-natural heterocyclic moiety that can participate in base pairing or base stacking interaction on an oligonucleotide strand.

“Moiety” and “group” are used interchangeably herein to refer to a portion of a molecule, typically having a particular functional or structural feature, e.g. a linking group (a portion of a molecule connecting two other portions of the molecule), or an ethyl moiety (a portion of a molecule with a structure closely related to ethane). A “triaryl methyl linker group” as used herein references a triaryl methyl group having one or more substituents on the aromatic rings of the triaryl methyl group, wherein the triaryl methyl group is bonded to two other moieties such that the two other moieties are linked via the triaryl methyl group. An “intermediate linking group” references any linking group adjacent to the triaryl methyl linker group and bound to the triaryl methyl linker group. “Linkage” as used herein refers to a first moiety bonded to two other moieties, wherein the two other moieties are linked via the first moiety. Typical linkages include ether (—O—), oxo (—C(O)—), amino (—NH—), amido (—N—C(O)—, thio (—S—), phospho (—P—), ester (—O—C(O)—.

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect. Depending on the context, “connected”, “linked”, or other like term indicates that two groups are bound to each other, wherein the attachment may be direct or indirect.

“Functionalized” references a process whereby a material is modified to have a specific moiety bound to the material, e.g. a molecule or substrate is modified to have the specific moiety; the material (e.g. molecule or substrate) that has been so modified is referred to as a functionalized material (e.g. functionalized molecule or functionalized substrate).

The term “halo” or “halogen” is used in its conventional sense to refer to a chloro, bromo, fluoro or iodo substituent.

By “protecting group” as used herein is meant a species which prevents a portion of a molecule from undergoing a specific chemical reaction, but which is removable from the molecule following completion of that reaction. This is in contrast to a “capping group,” which permanently binds to a segment of a molecule to prevent any further chemical transformation of that segment. A “hydroxylprotecting group” refers to a protecting group where the protected group is a hydroxyl. “Reactive site hydroxyl” references a hydroxyl group capable of reacting with an activated nucleotide monomer to result in an internucleotide bond being formed. In typical embodiments, the reactive site hydroxyl is the terminal 5′-hydroxyl during 3′-5′ polynucleotide synthesis and is the 3′-hydroxyl during 5′-3′ polynucleotide synthesis. An “acid labile protected hydroxyl” is a hydroxyl group protected by a protecting group that can be removed by acidic conditions. Similarly, an “acid stabile protected hydroxyl” is a hydroxyl group protected by a protecting group that is not removed (is stabile) under acidic conditions. An “acid labile linking group” is a linking group that releases a linked group under acidic conditions.

A trityl group is a triphenyl methyl group, in which one or more of the phenyl groups of the triphenyl methyl group is optionally substituted. A “substituted trityl group” or a “substituted triphenyl methyl group” is a triphenyl methyl group on which at least one of the hydrogens of the phenyl groups of the triphenyl methyl group is replaced by a substituent.

The term “substituted” as used to describe chemical structures, groups, or moieties, refers to the structure, group, or moiety comprising one or more substituents. As used herein, in cases in which a first group is “substituted with” a second group, the second group is attached to the first group whereby a moiety of the first group (typically a hydrogen) is replaced by the second group.

“Substituent” references a group that replaces another group in a chemical structure. Typical substituents include nonhydrogen atoms (e.g. halogens), functional groups (such as, but not limited to amino, sulfhydryl, carbonyl, hydroxyl, alkoxy, carboxyl, silyl, silyloxy, phosphate and the like), hydrocarbyl groups, and hydrocarbyl groups substituted with one or more heteroatoms. Exemplary substituents include alkyl, lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, boronyl, and modified lower alkyl.

A “group” includes both substituted and unsubstituted forms. Typical substituents include one or more lower alkyl, modified alkyl, any halogen, hydroxy, or aryl. Any substituents are typically chosen so as not to substantially adversely affect reaction yield (for example, not lower it by more than 20% (or 10%, or 5% or 1%) of the yield otherwise obtained without a particular substituent or substituent combination).

Hyphens, or dashes, are used at various points throughout this specification to indicate attachment, e.g. where two named groups are immediately adjacent a dash in the text, this indicates the two named groups are attached to each other. Similarly, a series of named groups with dashes between each of the named groups in the text indicates the named groups are attached to each other in the order shown. Also, a single named group adjacent a dash in the text indicates the named group is typically attached to some other, unnamed group. In some embodiments, the attachment indicated by a dash may be, e.g. a covalent bond between the adjacent named groups. In some other embodiments, the dash may indicate indirect attachment, i.e. with intervening groups between the named groups. At various points throughout the specification a group may be set forth in the text with or without an adjacent dash, (e.g. amido or amido-, further e.g. Trl or Trl-, yet further e.g. Cgp, Cgp- or -Cgp-) where the context indicates the group is intended to be (or has the potential to be) bound to another group; in such cases, the identity of the group is denoted by the group name (whether or not there is an adjacent dash in the text). Note that where context indicates, a single group may be attached to more than one other group (e.g. the triaryl methyl linker group, herein; further e.g. where a linkage is intended, such as linking groups).

The term “MALDI-MS” references matrix assisted laser desorption/ionization mass spectrometry, which entails methods of mass spectrometric analysis which use a laser as a means to desorb, volatize, and ionize an analyte. In MALDI-MS methods, the analyte is contacted with a matrix material to prepare the analyte for analysis. The matrix material absorbs energy from the laser and transfers the energy to the analyte to desorb, volatize, and ionize the analyte, thereby producing ions from the analyte that are then analyzed in the mass spectrometer to yield information about the analyte. A “MALDI sample plate” is a device that, when disposed in an operable relationship with a laser desorption ionization source of a MALDI mass spectrometer, can be used to deliver ions derived from an analyte on the device to the mass spectrometer for analysis to obtain information about the analyte. In other words, the term “MALDI sample plate” refers to a device that is removably insertable into a MALDI mass spectrometer and contains a substrate having a surface for presenting analytes for detection by the mass spectrometer. Other references may refer to a MALDI sample plate, as used herein, as a “target” or a “probe”.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present. At various points herein, a moiety may be described as being present zero or more times: this is equivalent to the moiety being optional and includes embodiments in which the moiety is present and embodiments in which the moiety is not present. If the optional moiety is not present (is present in the structure zero times), adjacent groups described as linked by the optional moiety are linked to each other directly. Similarly, a moiety may be described as being either (1) a group linking two adjacent groups, or (2) a bond linking the two adjacent groups: this is equivalent to the moiety being optional and includes embodiments in which the moiety is present and embodiments in which the moiety is not present. If the optional moiety is not present (is present in the structure zero times), adjacent groups described as linked by the optional moiety are linked to each other directly.

Accordingly, an embodiment in accordance with the invention is directed to a method of analyzing a polynucleotide using matrix assisted laser desorption/ionization mass spectrometry (MALDI-MS). The embodiment comprises obtaining the polynucleotide bound to a substrate via a linker moiety having a triaryl methyl linker group. The polynucleotide bound to the substrate is then contacted with a matrix material and analyzed by MALDI-MS. The MALDI-MS analysis includes directing laser radiation at the matrix material, thereby exciting the matrix material and causing cleavage of the linker moiety, and analyzing ions generated as a result of this excitation and cleavage process to provide information about the polynucleotide.

Obtaining the polynucleotide bound to the substrate may be accomplished in any manner that provides the polynucleotide bound to the substrate via a linker moiety having a triaryl methyl linker group. In an embodiment, the polynucleotide is synthesized on the substrate using previously reported synthesis methods, e.g. those reported in U.S. Pat. No. 6,222,030 to Dellinger et al., U.S. patent application Ser. No. 09/916,369 to Dellinger et al. (filed on Jul. 27, 2001), U.S. patent application Ser. No. 10/652,063 to Dellinger et al. (filed on Aug. 30, 2003). The synthesis of the polynucleotide may involve providing a functionalized substrate having a nucleotide monomer bound to the substrate via a triaryl methyl linker group and then synthesizing a polynucleotide using the nucleotide monomer bound to the substrate as a starting point for synthesis. Given the disclosure herein, one of ordinary skill will be able to obtain the functionalized substrate and synthesize the polynucleotide on the substrate to obtain the polynucleotide bound to the substrate via a triaryl methyl linker group.

In another embodiment, the polynucleotide is procured as a polynucleotide that is in solution (not immobilized on a substrate) and is contacted with a functionalized substrate to result in the polynucleotide bound to the substrate via a linker moiety having a triaryl methyl linker group. In such an embodiment, the functionalized substrate may have the triaryl methyl linker group bound to the substrate and a reactive group bound to the substrate via the triaryl methyl linker group. The reactive group is capable of reacting with a corresponding active group on the polynucleotide in solution, thereby immobilizing the polynucleotide on the substrate. Given the disclosure herein, one of ordinary skill will be able to obtain a functionalized substrate having a triaryl methyl linker group bound thereto and a reactive group bound to the functionalized substrate via the triaryl methyl linker group. In yet another embodiment, the polynucleotide is procured as a polynucleotide that is in solution (not immobilized on a substrate). In such an embodiment, the polynucleotide is functionalized to have a triaryl methyl linker group bound to the polynucleotide and a reactive group bound to the polynucleotide via the triaryl methyl linker group. In such an embodiment, the reactive group is capable of reacting with a corresponding active group on the substrate, thereby immobilizing the polynucleotide on the substrate. Given the disclosure herein, one of ordinary skill will be able to obtain a functionalized polynucleotide having a triaryl methyl linker group bound thereto and a reactive group bound to the functionalized polynucleotide via the triaryl methyl linker group. Any suitable reactive group capable of reacting with a corresponding active group may be used; various such groups are known in the art and may be employed by one skilled in the art given the disclosure herein.

The polynucleotide is bonded to the substrate via a linker moiety having a triaryl methyl linker group. The linker moiety includes a triaryl methyl linker group, which is covalently bound to the polynucleotide, e.g. directly bound or bound via an intermediate linking group. The triaryl methyl linker group is also covalently bound to the substrate, e.g. directly bound or bound via an intermediate linking group, such that the polynucleotide is bound to the substrate via the linker moiety and via any optional intermediate linking groups. The linker moiety thus comprises the triaryl methyl linker group and any intermediate linking group(s) bound to the triaryl methyl linker group. The exact structure of such intermediate linking groups is not essential to the invention, but, if present, they should provide a stable connection between the linker moiety and the substrate and/or polynucleotide. In this context, a stable connection is one that is not subject to cleavage under the conditions typically encountered during the practice of the invention. An intermediate linking group may be bonded to the adjacent triaryl methyl linker group at any position of the intermediate linking group available to bind to the adjacent triaryl methyl linker group. Similarly, an intermediate linking group may be bonded to the adjacent substrate at any position of the intermediate linking group available to bind to the adjacent substrate. Also, an intermediate linking group may be bonded to the adjacent polynucleotide at any position of the intermediate linking group available to bind to the adjacent polynucleotide. In typical embodiments, the intermediate linking groups are selected from alkyl and modified alkyl groups and combinations thereof. In certain embodiments, the intermediate linking group is a single non-carbon atom, e.g. —O—, or a single non-carbon atom with one or more hydrogens attached, e.g. —N(H)—. In an embodiment, the intermediate linking group is selected from optionally substituted lower alkyl. In another embodiment, the intermediate linking group is selected from optionally substituted ethoxy, propoxy, or butoxy groups.

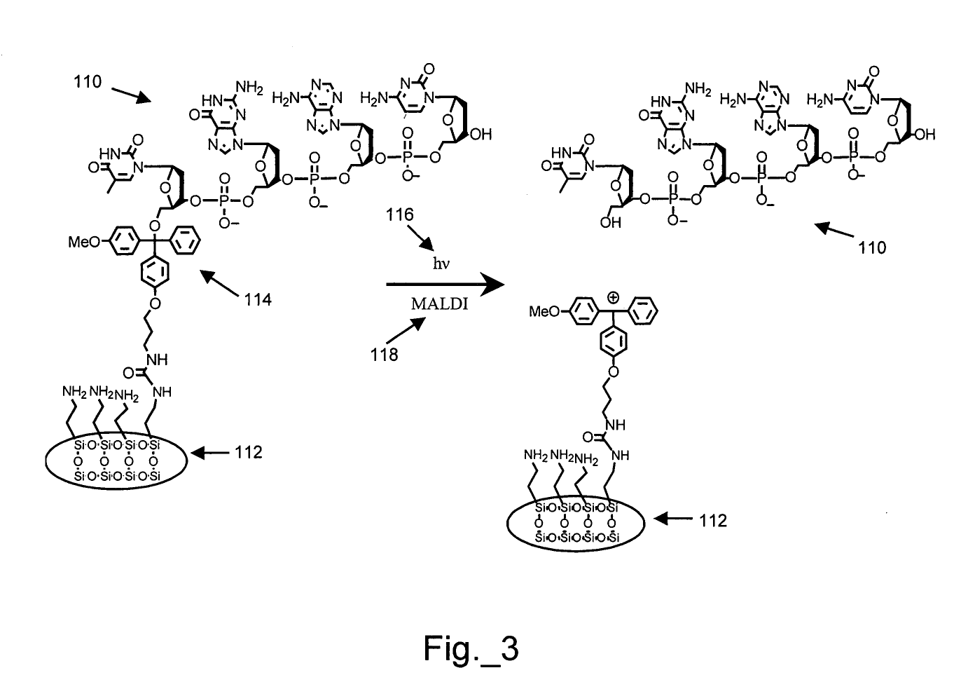

The linker moiety is characterized as being cleavable under the conditions of the MALDI-MS analysis to release the polynucleotide from the substrate. In particular embodiments, laser radiation directed at the matrix material (which is contacting the polynucleotide) results in cleavage of the linker moiety to release the polynucleotide from the substrate. Without being bound to any particular mechanism or limiting the invention in any way, it is believed that upon excitation of the matrix by the laser radiation, the triaryl methyl linker group of the linker moiety undergoes an acidic cleavage reaction at the central methyl carbon of the triaryl methyl group to result in a triaryl methyl cation and also to result in the polynucleotide being released from the substrate. At least some of the released polynucleotide will provide for ions that are analyzed by mass spectrometry to yield information about the polynucleotide. A typical reaction is shown in FIG. 3, in which a polynucleotide 110 bound to a substrate surface 112 via a triaryl methyl linker group 114 is subjected to laser radiation (“hv”) 116 during a matrix assisted laser desorption/ionization (“MALDI”) 118 process. The result of the reaction is that the polynucleotide 110 is released from the substrate surface 112. Ions derived from the polynucleotide that are desorbed and volatized may be analyzed in a mass spectrometer to yield information about the polynucleotide.

The polynucleotide, which is bonded to the substrate via the linker moiety having a triaryl methyl linker group, typically has at least 2, at least 5, or at least 10, and may have up to 20, up to about 100, up to about 200, or even more nucleotide subunits. In certain embodiments, the polynucleotide has 2, 3, 4, or 5 nucleotide subunits. In some embodiments, the polynucleotide may have appropriate protecting groups as are known in the art of polynucleotide synthesis to prevent or reduce undesired chemical reactivity. The polynucleotide typically includes naturally occurring and/or non-naturally occurring heterocyclic bases and may include heterocyclic bases which have been modified, e.g. by inclusion of protecting groups or any other modifications described herein, or the like. The polynucleotide is typically bound to the triaryl methyl linker group via a terminal 3′-O— or a 5′-O— of the polynucleotide, although any other suitable site is contemplated and is within the scope of the invention.

The invention provides a method of analyzing a polynucleotide using matrix assisted laser desorption/ionization mass spectrometry (MALDI-MS). In an embodiment, the method includes obtaining a composition having the structure (I)
●-Cgp-Trl-Cgp′-Pnt (I)
wherein the groups are defined as follows:
●- is a substrate,
Trl is a triaryl methyl linker group having three aryl groups, each bound to a central methyl carbon, at least one of said three aryl groups having one or more substituents,
Cgp is an intermediate linking group linking the substrate and the triaryl methyl linker group, or is a bond linking the substrate and the triaryl methyl linker group,
Pnt is a polynucleotide, and
Cgp′ is an intermediate linking group linking the polynucleotide and the triaryl methyl linker group, or is a bond linking the polynucleotide and the triaryl methyl linker group.
   The composition having the structure (I) is then contacted with a matrix material and analyzed by MALDI-MS. During the MALDI-MS analysis, laser radiation is directed at the matrix material, thereby exciting the matrix material and releasing the polynucleotide from the substrate. Ions generated as a result of this excitation and release process are then analyzed to provide information about the polynucleotide.

Obtaining the composition having the structure (I) may be accomplished in any manner that provides the polynucleotide bound to the substrate via the triaryl methyl linker group (and also via the intermediate linking groups, if present) as indicated in structure (I). In an embodiment, the polynucleotide is synthesized on the substrate using previously reported synthesis methods, e.g. those reported in U.S. Pat. No. 6,222,030 to Dellinger et al., U.S. patent application Ser. No. 09/916,369 to Dellinger et al. (filed on Jul. 27, 2001), U.S. patent application Ser. No. 10/652,063 to Dellinger et al. (filed on Aug. 30, 2003). The synthesis of the polynucleotide may involve providing a functionalized substrate having a nucleotide monomer bound to the substrate via a triaryl methyl linker group and then synthesizing a polynucleotide using the nucleotide monomer bound to the substrate as a starting point for synthesis. Given the disclosure herein, one of ordinary skill will be able to obtain the functionalized substrate and synthesize the polynucleotide on the substrate to obtain the composition having the structure (I).

In another embodiment, the polynucleotide is procured as a polynucleotide that is in solution (not immobilized on a substrate) and is contacted with a functionalized substrate to provide the polynucleotide bound to the substrate via the triaryl methyl linker group (and also via the intermediate linking groups, if present) as indicated in structure (I). In such an embodiment, the functionalized substrate may have the triaryl methyl linker group bound to the substrate and a reactive group bound to the substrate via the triaryl methyl linker group. The reactive group is capable of reacting with a corresponding active group on the polynucleotide in solution, thereby binding the polynucleotide to the substrate. Given the disclosure herein, one of ordinary skill will be able to obtain a functionalized substrate having a triaryl methyl linker group bound thereto and a reactive group bound to the functionalized substrate via the triaryl methyl linker group. In yet another embodiment, the polynucleotide is procured as a polynucleotide that is in solution (not immobilized on a substrate). In such an embodiment, the polynucleotide is functionalized to have a triaryl methyl linker group bound to the polynucleotide and a reactive group bound to the polynucleotide via the triaryl methyl linker group. In such an embodiment, the reactive group is capable of reacting with a corresponding active group on the substrate, thereby immobilizing the polynucleotide on the substrate. Given the disclosure herein, one of ordinary skill will be able to obtain a functionalized polynucleotide having a triaryl methyl linker group bound thereto and a reactive group bound to the functionalized polynucleotide via the triaryl methyl linker group. Any suitable reactive group capable of reacting with a corresponding active group may be used; various such groups are known in the art and may be employed by one skilled in the art given the disclosure herein.

Referring now to structure (I), the Cgp group is selected from (1) a linking group linking the substrate to the triaryl methyl linker group; or (2) a covalent bond between the substrate and the triaryl methyl linker group. In some embodiments in which Cgp is a linking group, Cgp is typically bound to a ring atom of one of the aryl groups of the triaryl methyl linker group, i.e. the Cgp group may be considered a substituent of one of the aryl groups of the triaryl methyl linker group. In other embodiments in which Cgp is a linking group, Cgp may be bound to the central methyl carbon of the triaryl methyl linker group. In some embodiments in which Cgp is a covalent bond, the substrate is typically bound to a ring atom of one of the aryl groups of the triaryl methyl linker group, i.e. the substrate may be considered a substituent of one of the aryl groups of the triaryl methyl linker group. In other embodiments in which Cgp is a covalent bond, Cgp may be bound to the central methyl carbon of the triaryl methyl linker group. In particular embodiments, the Cgp group may be any appropriate linking group (referenced herein as the Cgp linker group) that links the substrate and the triaryl methyl linker group, the Cgp linker group typically selected from (1) a lower alkyl group; (2) a modified lower alkyl group in which one or more linkages selected from ether-, oxo-, thio-, amino-, and phospho- is present; (3) a modified lower alkyl substituted with one or more groups including lower alkyl; aryl, aralkyl, alkoxyl, thioalkyl, hydroxyl, amino, sulfonyl, halo; or (4) a modified lower alkyl substituted with one or more groups including lower alkyl; alkoxyl, thioalkyl, hydroxyl, amino, sulfonyl, halo, and in which one or more linkages selected from ether-, oxo-, thio-, amino-, and phospho- is present. The Cgp linker group may be bonded to the adjacent triaryl methyl linker group at any position of the Cgp linker group available to bind to the adjacent triaryl methyl linker group. Similarly, the Cgp linker group may be bonded to the substrate at any position of the Cgp linker group available to bind to the substrate. In certain embodiments, the Cgp linker group is a single non-carbon atom, e.g. —O—, or a single non-carbon atom with one or more hydrogens attached, e.g. —N(H)—. In an embodiment, the Cgp linker group is selected from optionally substituted lower alkyl. In another embodiment, the Cgp linker group is selected from optionally substituted ethoxy, propoxy, or butoxy groups. The exact structure of the Cgp linker group is not essential to the invention, but, if present, the Cgp linker group should provide a stable connection between the triaryl methyl linker group and the substrate.

Again referring to structure (I), the Cgp′ group is selected from (1) a linking group linking the triaryl methyl linker group to the polynucleotide (typically at the terminal 5′-O or 3′-O of the polynucleotide, or other suitable site of the polynucleotide); or (2) a covalent bond between the triaryl methyl linker group and the polynucleotide (e.g. at the terminal 5′-O or 3′-O of the polynucleotide, or other suitable site of the polynucleotide). In some embodiments in which Cgp′ is a linking group, Cgp′ is typically bound to the central methyl carbon of the triaryl methyl linker group. In other embodiments in which Cgp′ is a linking group, Cgp′ may be bound to a ring atom of one of the aryl groups of the triaryl methyl linker group, i.e. the Cgp′ group may be considered a substituent of one of the aryl groups of the triaryl methyl linker group. In some embodiments in which Cgp′ is a covalent bond, the polynucleotide is typically bound to the central methyl carbon of the triaryl methyl linker group. In other embodiments in which Cgp′ is a covalent bond, Cgp′ may be bound to a ring atom of one of the aryl groups of the triaryl methyl linker group, i.e. the polynucleotide may be considered a substituent of one of the aryl groups of the triaryl methyl linker group. In particular embodiments, the Cgp′ group may be any appropriate linking group (referenced herein as the Cgp′ linker group) that links the triaryl methyl linker group to the polynucleotide, the Cgp′ linker group typically selected from (1) a lower alkyl group; (2) a modified lower alkyl group in which one or more linkages selected from ether-, oxo-, thio-, amino-, and phospho- is present; (3) a modified lower alkyl substituted with one or more groups including lower alkyl; aryl, aralkyl, alkoxyl, thioalkyl, hydroxyl, amino, sulfonyl, halo; or (4) a modified lower alkyl substituted with one or more groups including lower alkyl; alkoxyl, thioalkyl, hydroxyl, amino, sulfonyl, halo, and in which one or more linkages selected from ether-, oxo-, thio-, amino-, and phospho- is present. The Cgp′ linker group may be bonded to the adjacent triaryl methyl linker group at any position of the Cgp′ linker group available to bind to the adjacent triaryl methyl linker group. Similarly, the Cgp′ linker group may be bonded to the adjacent polynucleotide at any position of the Cgp′ linker group available to bind to the adjacent polynucleotide. In certain embodiments, the Cgp′ linker group is a single non-carbon atom, e.g. —O—, or a single non-carbon atom with one or more hydrogens attached, e.g. —N(H)—. In an embodiment, the Cgp′ linker group is selected from optionally substituted lower alkyl. In another embodiment, the Cgp′ linker group is selected from optionally substituted ethoxy, propoxy, or butoxy groups. The exact structure of the Cgp′ group is not essential to the invention, but, if present, it should provide a stable connection between the triaryl methyl linker group and the polynucleotide.

In certain embodiments, the polynucleotide has 2, 3, 4, or 5 nucleotide subunits. In some embodiments, the polynucleotide has at least 2, at least 5, or at least 10, and may have up to 20, up to about 100, up to about 200, or even more nucleotide subunits. The polynucleotide may have appropriate protecting groups as are known in the art of polynucleotide synthesis to prevent or reduce undesired chemical reactivity. The polynucleotide typically includes naturally occurring and/or non-naturally occurring heterocyclic bases and may include heterocyclic bases which have been modified, e.g. by inclusion of protecting groups or any other modifications described herein, or the like. The polynucleotide is typically bound to the triaryl methyl linker group of structure (I) via a terminal 3′-O— or a 5′-O— of the polynucleotide, although any other suitable site is contemplated and is within the scope of the invention.

The triaryl methyl linker group in the embodiments described herein typically has the structure (II) 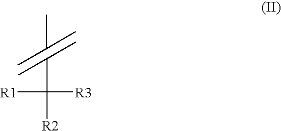
wherein the broken line represents a bond via which the triaryl methyl linker group is connected to the polynucleotide (e.g. directly or via an intermediate linking group). R1, R2, and R3 are independently selected from aromatic ring moieties (aryl groups), provided that one of R1, R2, and R3 is substituted by being bonded (e.g. directly or via an intermediate linking group) to the substrate. In other words, in a typical embodiment the substrate is bound to the central methyl carbon of the triaryl methyl linker group via one of R1, R2, and R3. Each aromatic ring moiety (aryl group) typically comprises one or more 4-, 5-, or 6-membered rings. Each aromatic ring moiety can independently be heterocyclic, non-heterocyclic, polycyclic or part of a fused ring system. Each aromatic ring moiety can be unsubstituted or substituted, e.g. substituted with one or more groups each independently selected from the group consisting of lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxylthio, mercapto, amino, imino, halo, cyano, nitro, nitroso, azido, carboxy, sulfide, sulfone, sulfoxy, phosphoryl, silyl, silyloxy, and boronyl; provided that one of R1, R2, and R3 is substituted by being bound to the substrate (e.g. directly or via an intermediate linking group). In an alternate embodiment, the broken line in structure (II) represents a bond via which the triaryl methyl linker group is connected to the substrate (e.g. directly or via an intermediate linking group), and one of R1, R2, and R3 is substituted by being bound to the polynucleotide (e.g. directly or via an intermediate linking group); in such embodiments, cleavage of the linker results in the release of the triaryl methyl group from the substrate. In other words, in such embodiments the polynucleotide is bound to the central methyl carbon of the triaryl methyl linker group via one of R1, R2, or R3.

Typical triaryl methyl groups that may be employed in embodiments herein are described in U.S. Pat. No. 4,668,777 to Caruthers, again provided that, as noted above, one of R1, R2, and R3 is substituted by being bound to the substrate (or, in alternate embodiments, bound to the polynucleotide); use of such triaryl methyl groups in accordance with the present invention is within ordinary skill in the art given the disclosure herein. A substituted triaryl methyl group may have one substituent (i.e. a singly substituted triaryl methyl group) on one of the aromatic rings of the triaryl methyl group, or may have multiple substituents (i.e. a multiply substituted triaryl methyl group) on one or more of the aromatic rings of the triaryl methyl group. As used herein, an aromatic ring moiety may be referenced as an “aromatic ring structure”. As used herein, the “central methyl carbon” of a triaryl methyl group is the carbon bonded directly to the three aromatic ring structures.

In certain embodiments, R2 and R3 are each independently selected from substituted or unsubstituted aromatic groups such as phenyl, biphenyl, naphthanyl, indolyl, pyridinyl, pyrrolyl, thiophenyl, furanyl, annulenyl, quinolinyl, anthracenyl, and the like, and R1 is selected from substituted aromatic groups such as phenyl, biphenyl, naphthanyl, indolyl, pyridinyl, pyrrolyl, thiophenyl, furanyl, annulenyl, quinolinyl, anthracenyl, and the like. In some embodiments, at least one of R1, R2 and R3 is selected from substituted or unsubstituted aromatic groups other than phenyl such as naphthanyl, indolyl, pyridinyl, pyrrolyl, furanyl, annulenyl, quinolinyl, anthracenyl, and the like; in such embodiments zero, one, or two of R1, R2, and R3 are selected from substituted or unsubstituted phenyl, provided that, as noted above, one of R1, R2, and R3 is substituted by being bound to the substrate (e.g. directly or via an intermediate linking group), or, in alternate embodiments, by being bound to the polynucleotide.

In some embodiments, R1, R2, and R3 are independently selected from structure (III).

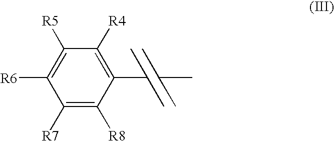

In structure (III), the broken line represents the bond to the central methyl carbon of the triaryl methyl linker group, and R4, R5, R6, R7, and R8 are each independently selected from hydrido, lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; provided that, for R3, one of R4, R5, R6, R7, and R8 denotes the linkage via which the triaryl methyl group is connected to one of the substrate or the polynucleotide (and the other of the substrate or polynucleotide is connected to the triaryl methyl linker group via the bond to the central methyl carbon denoted by the broken line in structure (II)).

In particular embodiments, R1, R2, and R3 are each independently selected from phenyl, methoxyphenyl, dimethoxyphenyl, and trimethoxyphenyl groups, such that the triaryl methyl linker group may be a trityl group, a monomethoxytrityl group, a dimethoxytrityl group, a trimethoxytrityl group, a tetramethoxytrityl group, a pentamethoxytrityl group, a hexamethoxytrityl group and so on; again provided as described above that one of R1, R2, and R3 is substituted by being bound (e.g. directly or indirectly) to one of the substrate or the polynucleotide (and the other of the substrate or polynucleotide is connected to the triaryl methyl linker group via the bond to the central methyl carbon denoted by the broken line in structure (II)).

In particular embodiments, R1, R2, and R3 are each independently selected from phenyl, methoxyphenyl groups, dimethoxyphenyl groups, trimethoxyphenyl groups, tetramethoxyphenyl groups, pentamethoxyphenyl groups, or furanyl groups such that the triaryl methyl linking group may be a substituted trityl group, a monomethoxytrityl group, a dimethoxytrityl group, a trimethoxyl trityl group, a tetramethoxy trityl group, a pentamethoxytrityl group, an anisylphenylfuranylmethyl group, a dianisylfuranylmethyl group, a phenyldifuranylmethyl group, an anisyldifuranylmethyl group or a trifuranylmethyl group, again provided as described above that one of R1, R2, and R3 is substituted by being bound (e.g. directly or indirectly) to one of the substrate or the polynucleotide (and the other of the substrate or polynucleotide is connected to the triaryl methyl linker group via the bond to the central methyl carbon denoted by the broken line in structure (II)).

The substrate typically comprises any material suitable for use in analysis of the polynucleotide using MALDI-MS. The material should be relatively (compared to the matrix and the polynucleotide) inert to the conditions used during the MALDI-MS analysis, e.g. exposure to laser radiation, temperature, reduced pressure, electric fields, matrix materials, etc. Typical materials include at least one material selected from the group including, but not limited to, cross-linked polymeric materials (e.g. divinylbenzene styrene-based polymers), silica, glass, ceramics, metals, plastics, and the like, and combinations thereof.

The substrate typically has a plurality of discrete, addressable regions, each region for binding to a different polynucleotide for ionization and analysis by MALDI-MS. Typically, the number of addressable regions present on the substrate ranges from about 1 to about 400, up to about 1600 or more, for example as many as about 3000, 5000, 10,000 or more discrete addressable regions may be present on a single substrate. The substrate may also have features that serve to confine or locate the polynucleotide and/or other substances (e.g. matrix materials or other reagents) on the substrate. Such features may include wells or depressions on the surface of the substrate, or a hydrophobic (or hydrophilic) pattern on the surface, or a visible grid pattern. The configuration or pattern of such features may vary depending on the particular MALDI protocol being employed, the number of features present, the size and shape of the features present, etc. For example, the configuration of the features may be in a grid format or other analogous geometric or linear format or the like, e.g., similar to a conventional microtiter plate grid pattern; in certain embodiments the features are present in a non grid-like or non-geometric pattern.

In general, the substrate may be any shape, and the choice of shape is generally defined by the shapes acceptable to the mass spectrometer employed in the subject methods. In particular embodiments, the substrate may have a square, rectangular, or circular shape, with one or more discrete addressable regions with features arranged in a parallel, random, spiral, grid configuration or any other configuration that can be accommodated on a surface of the substrate.

Typically the substrate has a surface to which the polynucleotide is bound via the triaryl methyl linker group. In certain embodiments, the substrate comprises a solid support and a modification layer disposed on (or bound to, e.g. directly or indirectly) the solid support, and the triaryl methyl linker group is bound to (e.g. directly or indirectly) the modification layer. Such modification layer may be formed on the substrate by methods known in the art to modify the surface properties of the solid support. The solid support typically comprises the same or similar materials or combinations of materials used to describe the substrate herein. In certain embodiments, the modification layer may be, e.g. a coating, a material deposited by deposition techniques known in the art, a hydrophobic layer, or a hydrophilic layer. In particular embodiments, the modification layer comprises a silane group, to which the triaryl methyl linker group is bound, directly or indirectly, e.g. via any linking group effective to link the triaryl methyl linker group to the silane group and stable to the conditions used in the methods described herein. Particularly contemplated are modification layers taught in U.S. Pat. No. 6,258,454 to Lefkowitz et al. (2001), which describes a moiety bound to a substrate via a linking group attached to a silane group bound to the substrate.

Substrates in accordance with the present invention may be made using silane modified substrates such as are employed in the Lefkowitz '454 patent and modifications thereof. In such methods, an available reactive group attached (directly or indirectly, e.g. via a linking group) to the silane group on the substrate provides a site for further attachment to the substrate to occur. Methods of preparing substrates having triaryl methyl linker groups bound to the solid support are taught in U.S. patent application Ser. No. 10/652,063 to Dellinger et al., filed on Aug. 30, 2003. The resulting functionalized substrate may be used for in situ synthesis of a polynucleotide or to bind to a pre-synthesized polynucleotide, as explained herein. Selection and preparation of the substrate will be based on experimental design considerations, such as the desired available reactive group attached to the substrate, number of different polynucleotides to be analyzed, design considerations for facilitating deposition of reagents such as polynucleotide, matrix materials, or other reagents, etc. Such selection and preparation is within the skill of those in the art given the disclosure herein.

The substrate may also have features that serve to aid in the MS analysis, e.g. electrically conductive materials coating the surface of the substrate or forming a conductive pattern (such as a grid) on the substrate. In typical embodiments, the substrate is a MALDI sample plate. In general, MALDI sample plates with a plurality of fluid retaining structures are known and described in U.S. Patent Publication serial Nos. 20030057368, and 20030116707. For example, e.g., “anchor” sample plates that have hydrophobic and/or hydrophilic coatings (see, e.g., U.S. Pat. No. 6,287,872) are well known and purchasable in 96 sample and 384 sample formats from Bruker Daltonik (Germany). Other suitable MALDI sample plates are purchasable from Agilent Technologies (Palo Alto, Calif.).

In certain embodiments, the polynucleotide bound to a substrate via the triaryl methyl linker group may be obtained by a method comprising using a fluid delivery device to deliver reagents, analytes (e.g. polynucleotides), matrix materials, etc. to the substrate surface. The fluid delivery device may be, e.g. a pulse-jet fluid delivery device or a contact fluid delivery device. The fluid delivery device, in certain embodiments, may also be employed to perform in situ synthesis of the polynucleotides on the substrate surface. Suitable fluid delivery devices include pulse-jet printing devices, and contact printing devices such as pipetting robots, capillary printers, and the like. Suitable pipetting robots may be used to perform all of the steps described above. Typical examples of pipetting robots include the following systems: GENESIS™ or FREEDOM™ of Tecan (Switzerland), MICROLAB 4000™ of Hamilton (Reno, Nev.), QIAGEN 8000™ of Qiagen (Valencia, Calif.), the BIOMEK 2000™ of Beckman Coulter (Fullerton, Calif.) and the HYDRA™ of Robbins Scientific (Hudson, N.H.). In particular embodiments, pulse-jet printing devices such as piezoelectric devices may be used (see e.g., Li et al., J. Proteome Res. (2002) 1:537-547; Sloan et al., Molecular and Cellular Proteomics (2002) 490-499).

A substrate, e.g. a MALDI sample plate, obtained as described herein may be inserted into the MALDI source of a mass spectrometer and used to assess the polynucleotide bound to the sample plate surface via a triaryl methyl linker group. Accordingly, the invention provides a method for assessing a sample of polynucleotides bound to the substrate. In general the methods involve obtaining a substrate having one or more polynucleotides of interest bound to the substrate via a linker moiety comprising a triaryl methyl linker group, contacting the one or more polynucleotides bound to the substrate with a matrix material, and evaluating the one or more polynucleotides using MALDI mass spectrometry.

In certain embodiments, the same polynucleotide may be present in two or more different regions of the substrate (e.g. sample sites on a MALDI sample plate). Typically, a plurality of different polynucleotides are bound to the substrate, each polynucleotide bound at its own addressable region. In this case, the resulting substrate (e.g. MALDI sample plate) will usually contain a plurality of regions containing different polynucleotides to be analyzed. Each region is then contacted with the matrix material, which is allowed to dry to form crystals, e.g. thus forming a prepared MALDI sample plate containing analytes that is suitable for use in a MALDI mass spectrometer. In some embodiments, a plurality of polynucleotides are bound at the same addressable region of the substrate such that the plurality of polynucleotides are analyzed simultaneously in the mass spectrometer. In some embodiments, one or more of the addressable regions will not be bound to a polynucleotide.

Prior to analysis by mass spectrometry, the polynucleotide bound to the substrate is typically contacted with an energy absorbing matrix material, as is known in the art. The matrix material is typically a small organic acid compound with certain properties that facilitate the performance of MALDI. Accordingly, a matrix material is selected based on a variety of factors such as the analyte of interest (such as charge, type or size of molecule), and the like. For example, a matrix material is selected that allows the cleavage of the triaryl linker and release of the polynucleotide from the substrate. Further, a matrix material should be selected that provides for generation of a sufficient quantity of ions to be analyzed in a mass spectrometer to obtain information about the polynucleotide.

Examples of matrix materials include, but are not limited to, sinapinic acid (SA) and derivatives thereof, such as alpha-cyano sinapinic acid; cinnamic acid and derivatives thereof, such as 3,5-dimethoxy-4-hydroxycinnamic acid; 2,5-dihydroxybenzoic acid (DHB); and dithranol. Further examples of matrices that are typical for use with polynucleotide analytes include 3-hydroxy-picolinic acid (HPA); 2,4,6-trihydroxyacetophenone (246THAP); 4-hydroxy-3-methoxycinnamic acid (Ferulic acid); trans-Indole-3-acrylic acid (IAA); 2,3,4-trihydroxyacetophenone (234THAP); 4-hydroxy-alpha-cyano-cinnamic acid methyl ester. In some embodiments, mixtures of two or more of the materials listed in this paragraph (or yet other matrix materials known in the art) may be used as the matrix material in the methods of the present invention. In some embodiments the addition of compounds like trifluoroacetic acid (TFA) and ammonium citrate are used to increase the quantity of desired ions and/or suppress the formation of ions that do not give useful information for the analyte. The desired matrix material (or combination of matrix materials) is typically dissolved in a suitable solvent that is selected at least in part based on suitability for applying the matrix material to the substrate to gain good contact between the matrix material and the polynucleotide and the triarylmethyl linker group. For example, in the analysis of oligonucleotides, 3-hydroxy-picolinic acid (HPA) dissolved in a solvent of acetonitrile and water may be employed. After application of the matrix material to the substrate, e.g. contacting a site on the substrate having a polynucleotide bound thereto, the matrix material is allowed to dry to form crystals.

The polynucleotide may be analyzed using any mass spectrometer that has the capability of measuring masses with a desired mass range, level of mass accuracy, precision, and resolution. Accordingly, the polynucleotides may be analyzed by any one of a number of mass spectrometry methods, including, but not limited to, matrix-assisted laser desorption ionization time-of-flight mass spectrometry (MALDI-TOF) and any tandem MS such as QTOF, TOF-TOF, etc. The mass spectrometry protocol may be an atmospheric pressure (AP) MALDI protocol or a vacuum MALDI protocol. Mass spectrometry methods are generally well known in the art (see Burlingame et al. Anal. Chem. 70:647R-716R (1998); Kinter and Sherman, Protein Sequencing and Identification Using Tandem Mass Spectrometry Wiley-Interscience, New York (2000)). Any convenient MALDI protocol may be adapted and employed with the subject invention. Representative MALDI protocols, as well as apparatuses for use in performing MALDI protocols, that may be adapted for use with the subject invention include, but are not limited to, those described in International Publication Nos.: GB 2,312782 A; GB 2,332,273 A; GB 2,370114A; and EP 0964427 A2, as well as in U.S. Patent Publication No. 2002031773; and U.S. Pat. Nos. 5,498,545; 5,643,800; 5,777,324; 5,777,860; 5,828,063; 5,841,136; 6,111,251; 6,287,872; 6,414,306; and 6,423,966 The basic processes associated with a mass spectrometry method are the generation of gas-phase ions derived from the sample, and the measurement of their mass. The analysis by MALDI-MS typically provides information about the polynucleotides, such as the mass of the isolated analytes or fragments thereof, and their relative or absolute abundances in the sample, information about identity of the polynucleotide, etc.

The analysis by MALDI-MS typically includes evaluation of the data obtained from mass spectrometry analysis. For example, molecular mass data may be compared against expected values for known or anticipated analytes. The evaluation of the molecular mass data may involve the elimination of signals obtained that are not derived from the analytes of interest, so that only those signals corresponding to the predetermined analytes may be retained. In many embodiments, the masses of the analytes or fragments thereof are stored in a table of a database and the table usually contains at least two fields, one field containing molecular mass information, and the other field containing analyte identifiers, such as names or codes. As such, the subject methods may involve comparing data obtained from mass spectrometry to a database to identify data for an analyte of interest. In general, methods of comparing data produced by mass spectrometry to databases of molecular mass information to facilitate data analysis is very well known in the art (see, e.g., Yates et al., Anal. Biochem. (1993) 214:397-408; Mann et al., Biol Mass Spectrom. (1993) 22:338-45; Jensen et al., Anal. Chem. (1997) D69:4741-50; and Cottrell et al., Pept Res. 1994 7:115-24) and, as such, need not be described here in any further detail. Accordingly, information, e.g., data, regarding the amount of analytes in a sample of interest (including information on their presence or absence) may be obtained using mass spectrometry.

As is well known in the art, for each analyte, information obtained using mass spectrometry may be qualitative (e.g., showing the presence or absence of an analyte, or whether the analyte is present at a greater or lower amount than a control analyte or other standard) or quantitative (e.g., providing a numeral or fraction that may be absolute or relative to a control analyte or other standard). Also as is known, standards for assessing mass spectrometry data may be obtained from a control analyte that is present in the isolated analytes, such as an analyte of known concentration, or an analyte that has been added at a known amount to the isolated analytes, e.g., a spiked analyte. Accordingly, the data produced by the subject methods may be “normalized” to an internal control, e.g. an analyte of known concentration or the like.

By comparing the results from assessing the presence of an analyte in two or more different samples using the methods set forth above, the relative levels of an analyte in two or more different samples may be obtained. In other embodiments, by assessing the presence of at least two different analytes in a single sample, the relative levels of the analytes in the sample may be obtained.

In typical embodiments, a polynucleotide is analyzed by mass spectrometry, and, by integrating the signals produced by the ions derived from the polynucleotide, measurements corresponding to the abundance of particular ions are provided. Using software that is already available and commonly used to identify ion masses, the data is usually compared to a database of ion masses expected for the polynucleotides. By doing this comparison, the identity and abundance of the polypeptide corresponding to a particular ion becomes known. Depending on the exact method used, a table containing data on the abundance of ions (or the corresponding polynucleotides) may be exported to a separate database, and saved.

Methods in accordance with the current invention may be employed in a variety of diagnostic, drug discovery, and research applications that include, but are not limited to, diagnosis or monitoring of a disease or condition (where analytes that are markers for the disease or condition are assessed), discovery of drug targets (an analyte whose level is modulated in a disease or condition is a drug target), drug screening (where the effects of a drug are monitored by assessing the levels of analytes), and research (where is it desirable to know the relative concentrations of a number of analytes in a sample, or, conversely, the relative levels of an analyte in two or more samples). In an embodiment, the method may be used to assess in situ synthesis of a polynucleotide on a substrate, e.g. to determine identity, yield, or purity of product, or other quality control measure. In another embodiment, the method may be used to assess deposition of a polynucleotide on a substrate (to determine identity, yield, purity, etc.).

### EXAMPLES

The practice of the present invention will employ, unless otherwise indicated, conventional techniques of synthetic organic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, percents are wt./wt., temperature is in ° C. and pressure is at or near atmospheric. Standard temperature and pressure are defined as 20° C. and 1 atmosphere.

Abbreviations used in the examples include: THF is tetrahydrofuran; TLC is thin layer chromatography; HEX is hexane; Et₃N is triethylamine; MW is molecular weight; AcCN is acetonitrile; sat'd is saturated; EtOH is ethanol; B is a heterocyclic base having an exocyclic amine group, B^{Prot} is a heterocyclic base having an exocyclic amine group with a trityl protecting group on the exocyclic amine group; TiPSCI is 1,3-dichloro-1,1,3,3-tetraisopropyldisiloxane; TEMED is N,N,N′,N′-Tetramethylethylenediamine; Py is pyridine; MeCN is acetonitrile; DMT is dimethoxytrityl; MMT is monomethoxytrityl; TMT is trimethoxytrityl; Cyt^{DMT} is cytosine which has a dimethoxytrityl protecting group on the exocyclic amine group; Cyt^{TMT} is cytosine which has a trimethoxytrityl protecting group on the exocyclic amine group (and so on for other bases and protecting groups on the exocyclic amine group of the indicated base); MS is mass spectrometry, MS (ES) is mass spectrometry (electrospray), HRMS (FAB) is high resolution mass spectrometry (fast atom bombardment); DCM is methylene chloride; EtOAc is ethyl acetate; ⁱPr is isopropyl; Et₃N is triethylamine; TCA is trichloroacetic acid; TEAB is tetraethylammonium bicarbonate.

A synthesis of reagents used in certain embodiments of the present invention is now described. It will be readily apparent that the reactions described herein may be altered, e.g. by using modified starting materials to provide correspondingly modified products, and that such alteration is within ordinary skill in the art. Given the disclosure herein, one of ordinary skill will be able to practice variations that are encompassed by the description herein without undue experimentation.

The triaryl methyl linker can be synthesized as a phosphoramidite (e.g. step 6, below) and reacted with a hydroxyl containing surface of a substrate (e.g. by inkjet deposition of the linker phosphoramidite onto the surface of the substrate) to produce the cleavable linker bound to the substrate at any number of sites on the substrate (see equation designated reaction (XII), below).

### 4-Hydroxy-4′-Methoxytrityl Alcohol Step 1

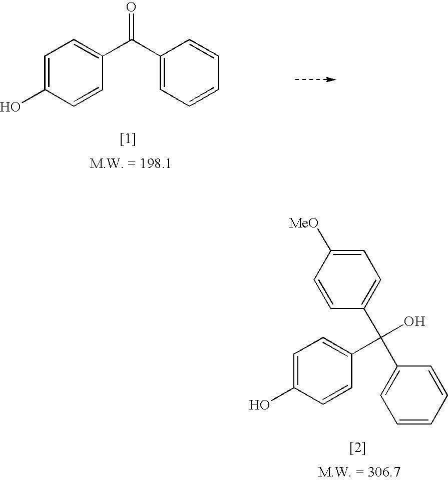 [0095] (A) 25.0 g (126.2 mmoles) 4-hydroxy Benzophenone (1); Aldrich # H2020-2
(B) 500 ml THF; Aldrich # 49446-1
(C) 700 ml of a 0.5 M Solution in THF (175 mmoles) 4-Anisyl Magnesium Bromide; Alpha-Aesar # 89435
   TLC System: HEX/EtOAc/Acetone (4:1:1)+0.5% Et₃N on silica gel

Using a 3-L 3-neck round bottom flask with a mechanical stirrer, U-tube thermometer and drying tube, (A) was added to (B) and the solution was cooled to 4° C. in a dry-ice/acetone bath, under Argon atmosphere. (C) was added drop wise over a period of 1 hour. Precipitate forms tan pink color. The temperature was kept between 0-5° C. during the addition. The mixture was removed from the bath and stirred at ambient temperature (under Argon atmosphere) for 16-hours. The solvent was evaporated in vacuo. The residue was suspended in 300 ml ether and 200 mL cold water. The ether layer was extracted with 150 mL saturated NaHCO₃ and 150 mL saturated NaCl and dried with MgSO₄. The solvent was evaporated, and 66 g of an oily residue was obtained. The residue was dissolved in 50 mL DCM, 30 g silica gel added and column purified over silica gel, with DCM/AcCN (19:1) as the initial mobile phase, changing to DCM/AcCN (9:1) as mobile phase for elution of the product. The product was column purified a second time over silica gel using EtOAc/HEX (1:1) as mobile phase for elution of the product.
Theoretical Yield: 38.6 g
Actual Yield: 23.9 g [62%]

¹H NMR (CDCl₃) 3.78 (3H, s), 6.75 (2H, d, J=8.8), 6.83 (2H, d, J=8.8), 7.11 (2H, d, J=8.8), 7.17 (2H, d, J=8.8), 7.25-7.32 (5H, m); MS (ESI−) m/z 305 (M−1, 100); (ESI+) m/z 635 (M₂+Na, 33), 289 (M−H₂O, 100)

### 4-((3-Propoxy)-tert-Butyldimethylsilane)-4′-Methoxytrityl Alcohol Step 2

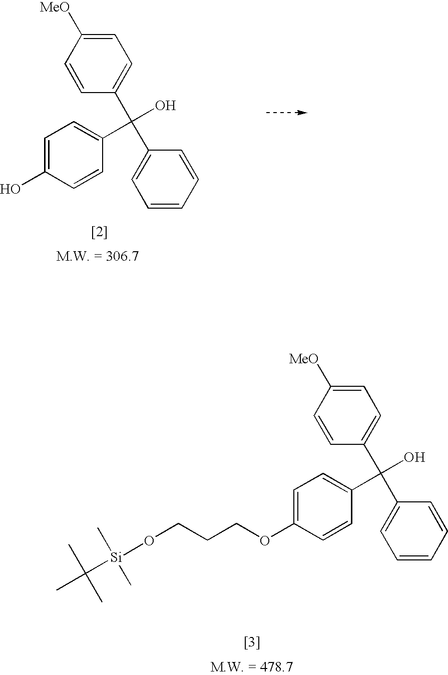 TLC System: DCM/AcCN [19:1]
(A) 24.0 g (78.0 mmoles) [2]
(B) 21.6 g (156 mmoles) potassium carbonate MW=138.1; Aldrich # 20961-9
(C) 60 g (235 mmoles) (3-Bromopropoxy)-tert-butyldimethylsilane MW=253.3; Aldrich # 42,906-6
(D) Single (Dry) Crystal Potassium Iodide MN 166.1; Aldrich # 22194-5
(E) 600 mL Toluene

Using a 2 L 3-neck round bottom flask equipped with a thermometer, reflux condenser, drying tube and stir bar, (A), (B) (C), and (D) were added to (E) in sequential order. The mixture was heated to reflux for 24 hours. The solvent was evaporated. The residue was partitioned between 750 mL DCM and 300 mL water. The DCM layer was washed twice with 400 mL sat'd NaCl then dried over MgSO₄.
Theoretical Yield: 37.3 g
Actual Yield: 16 g [143%]

MS (FAB+) m/z 479, 462 (M-OH, 100)

### 4-((3-Propoxy)-tert-Butyldimethylsilane)-4′-Methoxytrityl Chloride Step 3

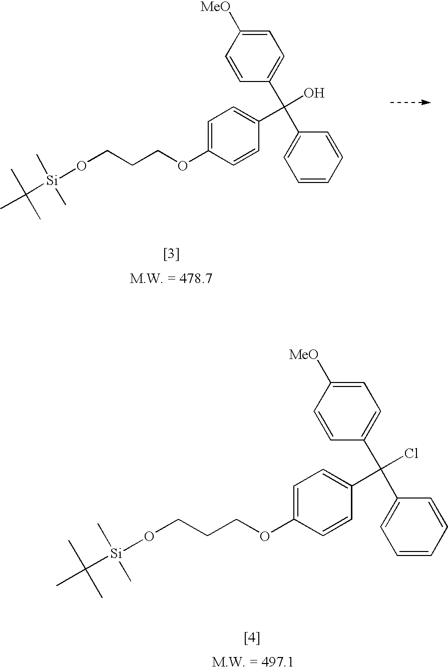 TLC System: Hexane/EtOAc [2:1]
(A) 5.0 g (10.44 mmol) [3]
(B) 18.2 mL (208 mmol) oxalyl chloride MW=126.9; Aldrich # 32042-0
(C) 150 mL Hexane

A 250 mL 3-neck round bottom flask was equipped with a cold-finger reflux/distillation condenser, magnetic stir bar, and two silicon rubber septa. (A) was suspended in (C) in the flask, and the flask was placed under argon and stirred. (B) was added to the stirring solution drop wise. Upon addition the suspended material dissolved and small bubbles formed in the flask. The reaction was refluxed overnight. The next morning the refluxing reaction consisted of a clear refluxing solution and a viscous orange-red oil on the bottom of the flask. The condenser was then set to distill and the hexanes and excess (B) removed by distillation. The remaining oil was placed under high vacuum resulting in 6.7 g of a foamed solid, used in the following reaction.
Theoretical Yield: 5.2 g
Actual Yield 5.2 g [100%]

### 3′-O-(4-Chlorophenyl)-Carbonyl-2′-Deoxythymidine

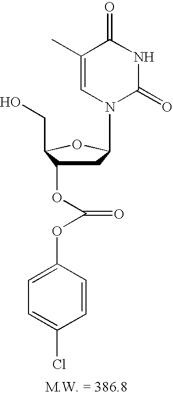

5′-O-(4,4′-Dimethoxytrityl)-2′-deoxythymidine (10.89 g, 20.0 mmol) was coevaporated from pyridine (3×40 mL), dissolved in pyridine (180 mL), and 4-chlorophenyl chloroformate (3.06 mL, 24.0 mmol) added with vigorous stirring. The mixture was stirred for 2 hours, solvent removed in vacuo, and the oily residue coevaporated with toluene (100 mL). The resulting oil was dissolved in dichloromethane (500 mL), extracted with sat. NaHCO₃ (250 mL) and brine (250 mL), dried over MgSO₄, and solvent evaporated to yield a viscous yellow oil. Purification by silica gel chromatography (0-2% ethanol in 100:0.1 dichloromethane:triethylamine) yielded 3′-O-(4-chlorophenyl)-carbonyl-5′-O-(4,4′-dimethoxytrityl)-2′-deoxythymidine as a white, glassy solid (10.93 g, 78.2%).

Anal. ¹H NMR (400 MHz, CDCl₃) δ 9.27 (1H, s, H₃), 7.63 (1H, s, H₆), 6.85-7.42 (17H, m), 6.54 (1H, m, H_{1′}), 5.43 (1H, m, H_{3′}), 4.32 (1H, m, H_{4′}), 3.78 (6H, s), 3.44-3.59 (2H, m, H_{5′}), 2.47-2.68 (2H, m, H₅′,_{5″}), 1.40 (3H, s); ¹³C NMR (100.5 MHz, CDCl₃) δ 163.7, 158.8, 152.7, 149.7, 149.2, 144.1, 135.1, 135.0, 131.7, 130.1, 130.0, 129.8, 129.6, 128.0, 127.2, 122.2, 113.3, 111.7, 87.3, 84.3, 83.6, 79.9, 63.6, 55.2, 37.8, 11.6; MS (FAB+) m/z 698 (M, 100).

To 3′-O-(4-chlorophenyl)-carbonyl-5′-O-(4,4′-dimethoxytrityl)-2′-deoxythymidine (2.50 g, 3.58 mmol) was added a 3% solution of trichloroacetic acid in dichloromethane (400 mL) with vigorous stirring. The mixture was stirred for 3 min before pyridine/methanol (1:1) was added drop wise until the red color of the DMT cation was quenched. The mixture was extracted with saturated NaHCO₃ (300 mL) and brine (300 mL), dried over MgSO₄, and solvent removed in vacuo. Purification of the resulting oil by silica gel chromatography (0-6% ethanol in dichloromethane) afforded the 3′-O-(4-Chlorophenyl)-Carbonyl-2′-Deoxythymidine as a white powder (1.30 g, 92%);

Anal. Calcd. for C₁₇H₁₇ClN₂O₇: C, 51.5; H, 4.3; N, 7.1. Found: C, 51.3; H, 4.5; N, 7.0. ¹H NMR (400 MHz, CDCl₃/d₄-MeOH) 9.57 (1H, s, H₃), 7.44 (1H, s, H₆), 7.25 (2H, d, J=8.8), 7.03 (2H, d, J=8.8), 6.17 (1H, m, H_{1′}), 5.27 (1H, m, H_{3′}), 4.17 (1H, m, H_{4′}), 3.83 (2H, m, H_{5′}), 2.42 (2H, m, H_{2′,2″}), 1.80 (3H, s); ¹³C NMR (100.5 MHz, CDCl₃/d₄-MeOH) 164.1, 152.8, 150.6, 149.2, 136.7, 131.7, 129.6, 122.2, 111.4, 86.3, 84.8, 79.5, 62.4, 37.0, 12.5; MS (ESI+) m/z 397 (M+1, 100).

### 5′-O-4-((3-Propoxy)-tert-Butyldimethylsilane)-4″-Methoxytrityl-3′-O-(4-Chlorophenyl)-Carbonyl-2′-Deoxythymidine Step 4

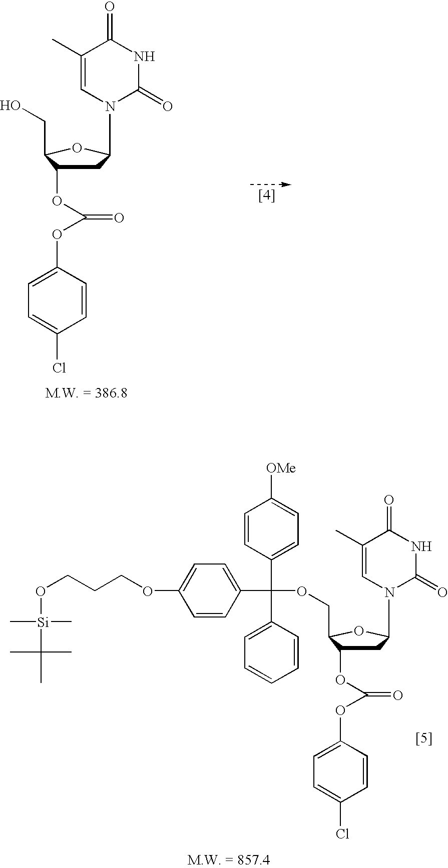

To 3′-O-(4-chlorophenyl)-carbonyl-2′-deoxythymidine (1.2 g, 3.1 mmol) in pyridine (35 mL) was added 4-((3-Propoxy)-tert-Butyldimethylsilane)-4′-Methoxytrityl Chloride (1.86 g, 3.75 mmol). The mixture was stirred for 4 h at which point the solvent was removed under reduced pressure. The residue was dissolved in dichloromethane, washed with 5% sodium carbonate and brine, dried (MgSO₄), and solvent removed in vacuo to yield a pale yellow oil. The 5′-O-4-((3-Propoxy)-tert-Butyldimethylsilane)-4″-Methoxytrityl-3′-O-(4-Chlorophenyl)-Carbonyl-2′-Deoxythymidine was isolated by silica gel chromatography using 1-4% methanol/dichloromethane as eluant as a pale yellow glassy solid (2.4 g, 90.0%); MS (FAB+) m/z 743 (M, 100).

### 5′-O-4-(3-Hydroxypropyl)-4″-Methoxytrityl-3′-O-(4-Chlorophenyl)-Carbonyl-2′-Deoxythymidine Step 5

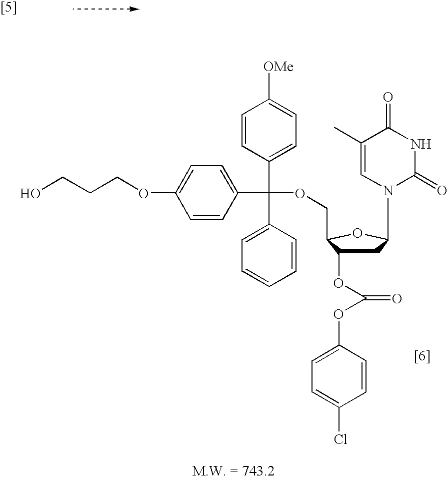

5′-O-4-((3-Propoxy)-tert-butyldimethylsilane)-4″-methoxytrityl-3′-O-(4-chlorophenyl)-carbonyl-2′-deoxythymidine (2.4 g, 2.8 mmol) was dissolved in anhydrous pyridine (75 mL) using a magnetic stirrer. The flask was kept anhydrous under argon and cooled in an ice/water bath. Hydrogen fluoride pyridine (100 μL) Fluka cat# 47586 was dissolved in 10 mL of anhydrous pyridine and added to the stirring flask. The reaction was allowed to stir for 30 min then evaporated to a rust brown oil. The residue was dissolved in dichloromethane, washed with 5% sodium carbonate and brine, dried (MgSO₄), and solvent removed in vacuo to yield a dark yellow oil. The 5′-O-4-(3-Hydroxypropyl)-4″-Methoxytrityl-3′-O-(4-Chlorophenyl)-Carbonyl-2′-Deoxythymidine was isolated by silica gel chromatography using 0-3% methanol/dichloromethane as eluant as a pale yellow glassy solid (2.4 g, 90.0%); MS (FAB+) m/z 859 (M, 100).

### 5′-O-4-(3-propyloxy(2-Cyanoethyl N,N-diisopropylphosphoramidite))-4′-Methoxytrityl-3′-O-(4-Chlorophenyl)-Carbonyl-2′-Deoxythymidine Step 6

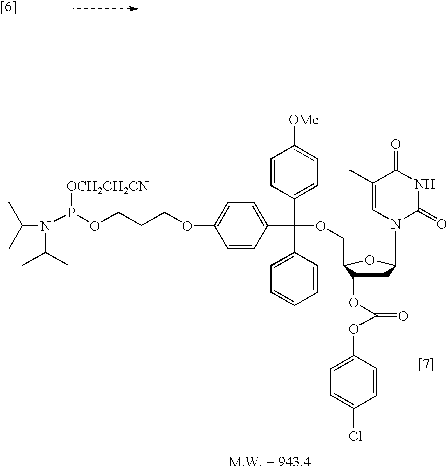

5′-O-4-(3-Hydroxypropyl)-4″-Methoxytrityl-3′-O-(4-Chlorophenyl)-Carbonyl-2′-Deoxythymidine 3.7 g (5.0 mmol) and tetrazole (175 mg, 2.50 mmol) were dried under vacuum for 24 h then dissolved in dichloromethane (100 mL). 2-Cyanoethyl N,N,N′,N′-tetraisopropylphosphorodiamidite (2.06 mL, 6.50 mmol) was added in one portion and the mixture stirred over 1 h. The reaction mixture was washed with sat. NaHCO₃ (150 mL) and brine (150 mL), dried over MgSO₄, and applied directly to the top of a silica column equilibrated with hexanes. The dichloromethane was flashed off the column with hexanes, and the product eluted as a mixture of diastereoisomers using 1:1 hexanes:ethyl acetate then ethyl acetate. After evaporation of solvents in vacuo and coevaporation with dichloromethane, product was isolated as friable, white, glassy solids in 75% yield; ³¹P NMR (162.0 MHz, CDCl₃) 148.89, 148.85; MS (FAB+) m/z 945 (FAB−) m/z 943

It will be apparent to one of skill in the art that the series of syntheses described above may be altered to employ analogous starting materials that react in a similar manner to give analogous products, and that such alteration of the synthesis is within ordinary skill in the art. For example, thymidine may be replaced with N-4-dimethoxy trityl-2′-deoxycytidine in step 4 to give 5′-O-4-(3-propyloxy-(2-cyanoethyl N,N-diisopropyl-phosphoramidite))-4″-methoxytrityl-3′-O-(4-chlorophenyl)-carbonyl-N-4-dimethoxytrityl-2′-deoxycytidine as the final product. As another example, in step 2, the (3-bromopropoxy)-tert-butyldimethylsilane may be replaced with (4-bromobutoxy)-tert-butyldimethylsilane to give 4-((4-Butoxy)-tert-butyldimethylsilane)-4′-methoxytrityl alcohol the product of step 2. As another example, it will be appreciated that the nucleoside moiety may be bound to the triaryl methyl linker group via either the 3′-OH or the 5′-OH. Such a modification will be accomplished by reacting a 5′-O-protected nucleoside with the trityl linker under conditions that enhance the rate of trityl reaction with secondary hydroxyls such as the addition of an acylation catalyst like N,N-dimethlyaminopyridine or silver salts as well as other techniques well known to one skilled in the art.

Furthermore, in the reaction designated as “Step 1”, above, the starting materials may be modified to yield a product wherein one or more of the phenyl (or substituted phenyl) rings is replaced by an alternate aromatic ring moiety, such as substituted or unsubstituted aromatic groups such as phenyl, biphenyl, naphthanyl, indolyl, pyridinyl, pyrrolyl, thiophenyl, furanyl, annulenyl, quinolinyl, anthracenyl, and the like. Such products may then be used as alternative starting materials in the reaction designated “Step 2” (and so on through the rest of the described syntheses) to give a triaryl methyl-modified nucleotide monomer, above.

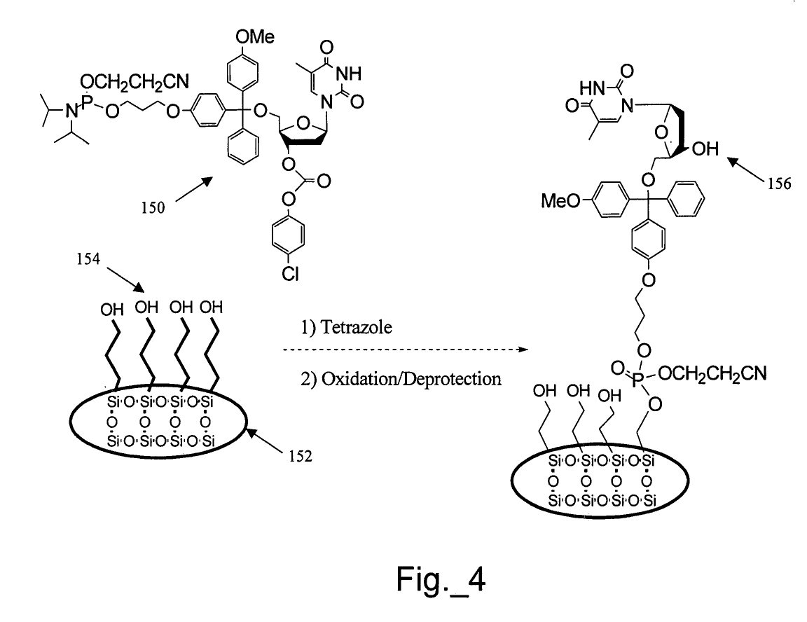

As shown in the reaction illustrated in FIG. 4, the 5′-linked molecules 150 can then be reacted with a substrate 152 having a reactive moiety 154 such as a hydroxyl group, thiol group, or amino group, wherein the substrate 152 is suitable for use for polynucleotide synthesis. The 3′-hydroxyl 156 of the nucleoside moiety may then be used as a starting point for performing cycles of a polynucleotide synthesis reaction to give a product in which a polynucleotide strand is bound to the substrate via the trityl group. An example of such a product is shown in FIG. 3 in which an oligonucleotide that is four nucleotides long has been synthesized and is bound to the substrate via the trityl moiety.

The reaction illustrated in FIG. 4 (or similar reactions apparent to those of ordinary skill given the disclosure herein) may be conducted at one or more regions of an array substrate, followed by cycles of a polynucleotide synthesis reaction at each region, to provide for one or more cleavable features of the fabricated array provided in accordance with the method of the present invention. Once the synthesis is complete, the substrate and the attached polynucleotide can be contacted with a matrix material and then subjected to analysis by MALDI-MS.
MALDI/TOF Analysis of Trityl Linker on Planar Glass Surface

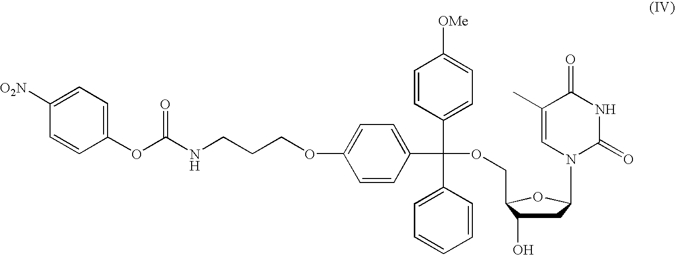

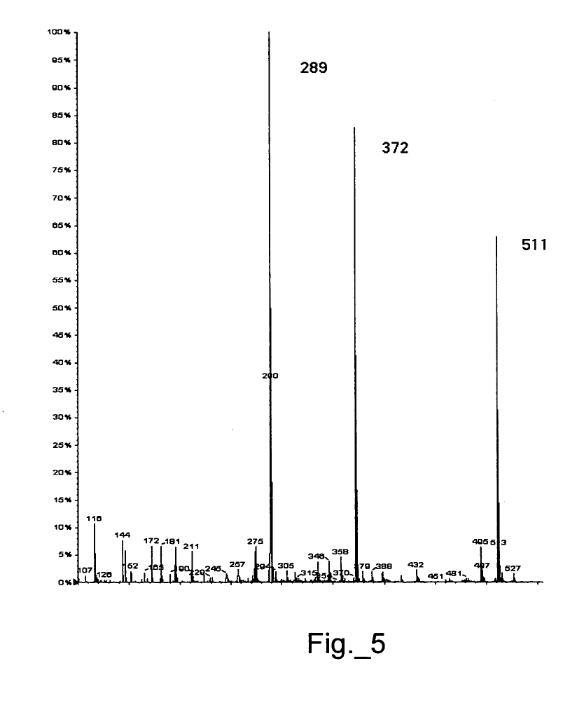

MALDI analysis of structure (IV) in positive ion mode with alpha-cyano-4-hydroxycinnamic acid, DHB or sinapinic acid as the matrix gives the mass spectra shown in FIG. 5. The prominent signals in FIG. 5 were assigned the following structures:

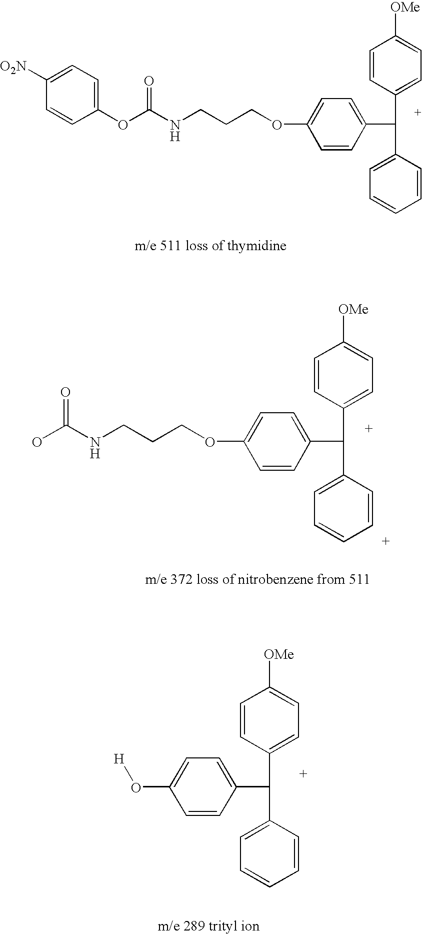

In negative ion mode no ions other than matrix ions were detected. Alpha-Cyano-4-Hydroxycinnamic Acid, DHB and sinipinic acid are not considered to be good matrices for generating negative ions. Further choices of MALDI matrices potentially capable of supporting negative ion generation include the compounds 3-HPA, 234THAP, 246THAP, and IAA.

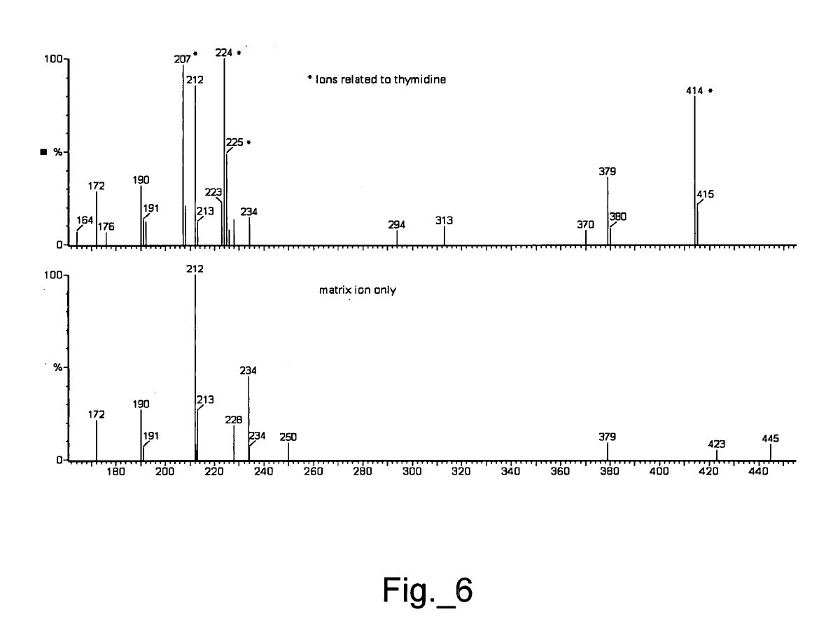

MALDI of the ALTA linker with thymidine (see FIG. 4) in positive ion mode using Alpha-Cyano-4-Hydroxycinnamic Acid as the matrix gives the mass spectra seen in FIG. 6. The prominent signals in FIG. 6 were assigned structures as follows: 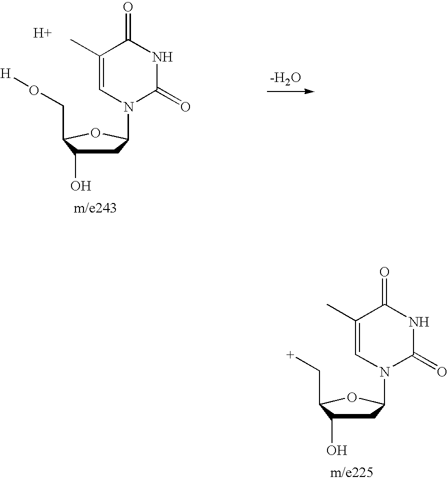
225 Thymidine with loss of water
414 Thymidine with loss of water with the addition of the matrix molecule
207 m/e 225 with the loss of an additional water molecule
   DHB and sinipinic acid could not be used as several of the matrix ions overlap with the masses of the ions from thymidine.

As can be seen from the solution data the formation of the Thymidine ions can only be a result of the dissociation of the linker attached to the glass plate. Otherwise the trityl group would carry the charge and the thymidine would not be observed in positive ion mode. Use of other matrices may give better signal to noise and well as fewer ions in the same mass range as the Thymidine.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

1. A method of analyzing a polynucleotide using matrix assisted laser desorption/ionization mass spectrometry, the method comprising
a) obtaining the polynucleotide bound to a substrate via a linker moiety, the linker moiety comprising a triaryl methyl linker group wherein the polynucleotide is bound to a substrate via the triaryl methyl linker group;
b) contacting the polynucleotide bound to the substrate with a matrix material; and
c) analyzing the polynucleotide by matrix assisted laser desorption/ionization mass spectrometry.

2. The method of claim 1, wherein obtaining the polynucleotide bound to the substrate via a linker moiety comprises synthesizing the polynucleotide on the substrate.

3. The method of claim 2, wherein synthesizing the polynucleotide on the substrate comprises providing a functionalized substrate having a nucleotide monomer bound to the substrate via the triaryl methyl linker group, and then synthesizing the polynucleotide using the nucleotide monomer bound to the substrate as a starting point for synthesizing the polynucleotide such that the resulting polynucleotide is bound to the substrate via the triaryl methyl linker group.

4. The method of claim 1, wherein obtaining the polynucleotide bound to the substrate via a linker moiety comprises procuring the polynucleotide in solution and contacting the polynucleotide in solution with a functionalized substrate to result in the polynucleotide bound to the substrate via the triaryl methyl linker group.

5. The method of claim 4, wherein the triaryl methyl linker group is bound to the functionalized substrate prior to contacting the polynucleotide in solution with the functionalized substrate.

6. The method of claim 4, wherein the triaryl methyl linker group is bound to the polynucleotide in solution prior to contacting the polynucleotide in solution with the functionalized substrate.

7. The method of claim 1, wherein the triaryl methyl linker group is covalently bound to the polynucleotide directly or via an intermediate linking group.

8. The method of claim 1, wherein analyzing the polynucleotide comprises directing laser radiation at the matrix material to generate ions including ions derived from the polynucleotide, and analyzing the ions in a mass spectrometer to provide information about the polynucleotide.

9. The method of claim 1, wherein the substrate is a mass spectrometer sample plate adapted to be disposed in operational relationship to a mass spectrometer to allow matrix assisted laser desorption/ionization analysis of the polynucleotide.

10. The method of claim 1, wherein the triaryl methyl linker group has the structure (II) 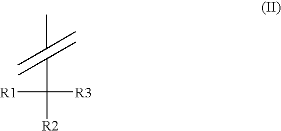
wherein the broken line represents a bond via which the triaryl methyl linker group is connected to the polynucleotide, and R1, R2, and R3 are independently selected from substituted or unsubstituted aryl groups, provided that one of R1, R2, and R3 is substituted by being bonded to the substrate.

11. The method of claim 1, wherein the triaryl methyl linker group has the structure (II) 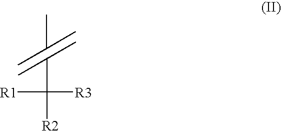
wherein the broken line represents a bond via which the triaryl methyl linker group is connected to the substrate, and R1, R2, and R3 are independently selected from substituted or unsubstituted aryl groups, provided that one of R1, R2, and R3 is substituted by being bonded to the polynucleotide.

12. A method of analyzing a polynucleotide using matrix assisted laser desorption/ionization mass spectrometry, the method comprising
a) obtaining a composition having the structure (I)
●-Cgp-Trl-Cgp′-Pnt (I)
wherein the groups are defined as follows:
●- is a substrate,
Trl is a triaryl methyl linker group having three aryl groups, each bound to a central methyl carbon, at least one of said three aryl groups having one or more substituents,
Cgp is a linking group linking the substrate and the triaryl methyl linker group, or is a bond linking the substrate and the triaryl methyl linker group,
Pnt is a polynucleotide, and
Cgp′ is a linking group linking the polynucleotide and the triaryl methyl linker group, or is a bond linking the polynucleotide and the triaryl methyl linker group.
b) contacting the composition having the structure (I) with a matrix material; and
c) analyzing the polynucleotide by matrix assisted laser desorption/ionization mass spectrometry.

13. The method of claim 12, wherein the triaryl methyl linker group has the structure (II) 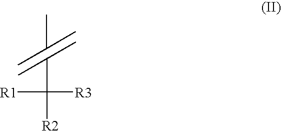
wherein the central methyl carbon is directly covalently bound to R1, R2, and R3, wherein R1, R2, and R3 are independently selected from substituted or unsubstituted aryl groups, provided that one of R1, R2, or R3 is substituted by being bound to one of the group consisting of the substrate and the polynucleotide.

14. The method of claim 13, wherein the broken line represents a bond via which the central methyl carbon is connected to the substrate, and the central methyl carbon is connected to the polynucleotide via one of R1, R2, or R3.

15. The method of claim 13, wherein the broken line represents a bond via which the central methyl carbon is connected to the polynucleotide, and the central methyl carbon is connected to the substrate via one of R1, R2, or R3.

16. The method of claim 13, wherein R1, R2, and R3 are independently selected from substituted phenyl and unsubstituted phenyl.

17. The method of claim 13, wherein R1, R2, and R3 are optionally substituted aryl groups independently selected from phenyl, biphenyl, naphthanyl, indolyl, pyridinyl, pyrrolyl, thiophenyl, furanyl, annulenyl, quinolinyl, and anthracenyl.

18. The method of claim 17, wherein at least one of R1, R2, and R3 is selected from naphthanyl, indolyl, pyridinyl, pyrrolyl, thiophenyl, furanyl, annulenyl, quinolinyl, and anthracenyl.

19. The method of claim 13, wherein R1, R2, and R3 are independently selected from phenyl, methoxyphenyl, dimethoxyphenyl, trimethoxyphenyl, and furanyl.

20. The method of claim 12, wherein the linking groups denoted Cgp and Cgp′ are independently selected from
(1) a lower alkyl group;
(2) a modified lower alkyl group in which one or more linkages selected from ether-, oxo-, thio-, amino-, phospho-, silyloxi, is present;
(3) a substituted lower alkyl group having one or more additional groups including lower alkyl, aryl, aralkyl, alkoxyl, thioalkyl, hydroxyl, amino, sulfonyl, halo; and
(4) a modified lower alkyl having (4a) one or more linkages selected from ether-, oxo-, thio-, amino-, phospho-, silyloxi and also having (4b) one or more additional groups selected from lower alkyl; aryl; aralkyl; alkoxyl; thioalkyl; hydroxyl; amino; nitro; nitroso; cyano; sulfonyl; carbonyl; carboxy; and halo.

21. The method of claim 12, wherein obtaining the composition having the structure (I) comprises synthesizing the polynucleotide on the substrate.

22. The method of claim 12, wherein obtaining the composition having the structure (I) comprises procuring the polynucleotide in solution and contacting the polynucleotide in solution with a functionalized substrate to result in the polynucleotide bound to the substrate via the triaryl methyl linker group.

23. The method of claim 12, wherein the Cgp′ group is a covalent bond linking the polynucleotide and the triaryl methyl linker group.

24. The method of claim 12, wherein analyzing the polynucleotide comprises directing laser radiation at the matrix material to generate ions including ions derived from the polynucleotide, and analyzing the ions in a mass spectrometer to provide information about the polynucleotide.

25. The method of claim 12, wherein the substrate is a mass spectrometer sample plate adapted to be disposed in operational relationship to a mass spectrometer to allow matrix assisted laser desorption/ionization analysis of the polynucleotide.
